(19) United States
(12) Patent Application Publication
(10) Pub. No.: US 2012/0310371 A1
(43) Pub. Date: Dec. 6, 2012

(54) RELEASABLE ATTACHMENT SYSTEM FOR A PROSTHETIC LIMB

(75) Inventors: Kent Bachus, Salt Lake City, UT (US); Daniel J. Triplett, Providence, UT (US); Trevor K. Lewis, Lehi, UT (US)

(73) Assignee: The University of Utah Research Foundation (UURF), Salt Lake City, UT (US)

(21) Appl. No.: 13/575,548

(22) PCT Filed: Jan. 28, 2011
(86) PCT No.: PCT/US11/23008

§ 371 Date: Jul. 26, 2012

Related U.S. Application Data
(60) Provisional application No. 61/299,868, filed on Jan. 29, 2010.

(51) Int. Cl. A61F 2/78

(52) U.S. Cl. 623/32

(57) ABSTRACT

Releasable prosthetic connectors are provided for use with prosthetic limbs and prosthetic mounting systems. The prosthetic connectors provide a secure, rigid connection between the prosthetic limb and the prosthetic mounting system under normal service loads. The prosthetic connectors provide safety release mechanisms which permit relative movement within the prosthetic connector when an excess load is experienced. The safety release mechanisms may be adjustable, and may include a warning system.

### BACKGROUND

1. Technical Field

The present disclosure relates to connectors, or couplers, which couple a prosthetic limb to a residual limb. The present disclosure presents prosthetic connectors which incorporate safety release mechanisms which permit the prosthetic limb to move relative to the residual limb in response to an excess load applied across the connector.

2. The Relevant Technology

A socket type prosthetic limb, such as a prosthetic arm or leg structure for use by amputees, is frequently constructed with an open-ended, and typically padded, socket structure for receiving and supporting the post-surgical stump of an amputated limb. By way of example, a socket type prosthetic leg may include such an open-ended socket structure at an upper end thereof for receiving and supporting the post-surgical upper leg of a transfemoral amputee. The socket structure may be permanently or releasably attached to the prosthetic leg with one of a variety of attachment systems, which may be referred to as residual limb attachment systems. Various straps and/or other fasteners may be provided for securing the prosthetic leg to the amputated limb to accommodate walking mobility at least on a limited basis. The mobility provided by such a prosthetic leg can be an important factor in both physical and mental rehabilitation of an amputee.

However, socket type prosthetic limbs are associated with a number of recognized limitations and disadvantages. In particular, the socket style prosthesis inherently couples mechanical loads associated with normal ambulatory activity through a soft tissue interface defined by the soft tissue covering the end or stump of the amputated limb, despite structural limitations of the soft tissue interface which limit its usefulness for this purpose. While many different arrangements and configurations of straps and other fasteners have been proposed for improved transmission and distribution of these mechanical loads to soft tissue structures to achieve an improved secure and stable prosthesis attachment, such arrangements have achieved only limited success. In addition, compressive loading of the stump soft tissue interface often results in blisters, sores, chafing and other undesirable skin irritation problems which have been addressed primarily by adding soft padding material within the socket structure. However, such soft padding material undesirably increases the extent of the soft or non-rigid interface between the amputated limb and prosthesis, in a manner that is incompatible with an optimally secure and stable prosthesis connection. As a result, particularly in the case of a prosthetic leg, traditional socket style connection structures and methods have generally failed to provide adequate stability for a normal walking motion without risking chronic soft tissue irritation problems.

More recently, external or exoskeletal prosthetic devices have been proposed, in which the external prosthesis is mechanically linked to the residual limb by means of a percutaneous bone anchored mounting system. These devices may also be described as direct skeletal attachment systems, and may be considered as another category of residual limb attachment system. In such devices, a rigid mounting post is surgically implanted and attached securely to patient bone by means of osseointegration or the like. The mounting post may, for example, be fitted into an intramedullary canal of a bone such as the femur or humerus. The mounting post extends from the bone attachment site and includes, or is attached to, a fixator pin, post, or other structure that protrudes through the overlying soft tissue at the end of the residual limb. Thus, one end of the bone anchored mounting system is rigidly secured to the patient's bone, and the other end is percutaneously exposed for secure and direct mechanical attachment to a prosthetic limb, or the like. The bone anchored mounting system provides a rigid linkage between the patient's bone and the external prosthetic limb.

In such bone anchored mounting systems, mechanical loads on the prosthetic limb during use are transmitted by the rigid linkage, through the fixator structure and mounting post, directly to patient bone. By mechanically linking and supporting the prosthesis directly from patient bone, amputees have reported a significant increase in their perception of the prosthesis as an actual and natural body part—a highly desirable factor referred to as “osseoperception.” Furthermore, bone anchored mounting systems significantly reduce compressive loads to the soft tissue at the end of the amputated limb, to correspondingly reduce the likelihood of blisters and other skin irritation problems. As a result, substantially improved and/or substantially normal patient movements are possible, and undesirable mechanical loading of the stump soft tissue is avoided.

Although a bone anchored mounting system may offer potentially dramatic improvements over a socket type prosthesis in terms of secure and stable prosthetic limb attachment and corresponding improvements in amputee lifestyle, major complications can arise in a bone anchored system when the prosthetic structure encounters a mechanical load that exceeds the strength of the prosthetic, the bone anchor system, its interface with the host bone, or the host bone itself. More particularly, in the event of an axial, bending, or torsion load that exceeds structural limitations, bending, cracking, fracture, or other types of failure can occur.

These failure modes represent traumatic and highly undesirable complications. Breakage of implanted structures such as the mounting post often requires surgical repair or revision. Breakage of the patient bone at or near the interface with the mounting post may also require surgical repair or reconstruction. Reseating or replacement of the mounting post may not be feasible after fracture of the host bone, thus forcing the amputee into an alternate residual limb attachment system, such as a socket type prosthesis, or eliminating the possibility of a prosthetic limb altogether.

There exists, therefore, a significant need for further improvements in and to prosthetic devices, particularly those that rely upon direct skeletal attachment. An improved attachment system securely couples the prosthetic limb to the residual limb in a manner that accommodates substantially normal patient movement and a corresponding normal range of mechanical loads, and includes a safety release mechanism adapted to release, or give way, in response to an excess mechanical load, thereby preventing transmission of the excess load to the residual limb, its soft tissues, a bone anchored mounting post, or other components of the system. The present disclosure sets forth various embodiments which fulfill these needs and provide further advantages.

### BRIEF DESCRIPTION OF THE DRAWINGS

Various embodiments of the present invention will now be discussed with reference to the appended drawings. It is appreciated that these drawings depict only example embodiments of the invention and are therefore not to be considered limiting of its scope.

FIG. 1A is a perspective view of a prosthetic connector; and FIG. 1B is an opposite perspective view of the prosthetic connector of FIG. 1A;

FIG. 2 is an exploded perspective view of the prosthetic connector of FIG. 1A;

FIG. 3A is a top view of the prosthetic connector of FIG. 1A; and FIG. 3B is a cross section view of the prosthetic connector of FIG. 1A taken along the section line 3B-3B indicated in FIG. 3A;

FIG. 4A is a perspective view of a sphere component of the prosthetic connector of FIG. 1A; FIG. 4B is an opposite perspective view of the sphere component of FIG. 4A; and FIG. 4C is a perspective view of the sphere component seated within a socket component of the prosthetic connector of FIG. 1A;

FIG. 5A is a perspective view of another prosthetic connector; FIG. 5B is an opposite perspective view of the prosthetic connector of FIG. 5A; FIG. 5C is a top view of the prosthetic connector of FIG. 5A; and FIG. 5D is a cross section view of the prosthetic connector of FIG. 5A taken along the section line 5D-5D indicated in FIG. 5C;

FIG. 6 is an exploded perspective view of the prosthetic connector of FIG. 5A;

FIG. 7A is a perspective view of yet another prosthetic connector; FIG. 7B is an opposite perspective view of the prosthetic connector of FIG. 7A; FIG. 7C is a top view of the prosthetic connector of FIG. 7A; and FIG. 7D is a cross section view of the prosthetic connector of FIG. 7A taken along the section line 7D-7D indicated in FIG. 7C;

FIG. 8 is an exploded perspective view of the prosthetic connector of FIG. 7A;

FIG. 9A is a perspective view of yet another prosthetic connector; FIG. 9B is an opposite perspective view of the prosthetic connector of FIG. 9A; FIG. 9C is a top view of the prosthetic connector of FIG. 9A; and FIG. 9D is a cross section view of the prosthetic connector of FIG. 9A taken along the section line 9D-9D indicated in FIG. 9C;

FIG. 10 is an exploded perspective view of the prosthetic connector of FIG. 9A; and

FIG. 11A is a perspective view of yet another prosthetic connector; FIG. 11B is a perspective view of a sphere component of the prosthetic connector of FIG. 11A with installed ball plungers; and FIG. 11C is a perspective view of a socket component of the prosthetic connector of FIG. 11A.

### DETAILED DESCRIPTION OF THE PREFERRED EMBODIMENTS

The present disclosure sets forth various embodiments of prosthetic connectors. It is appreciated that the systems and methods described herein may be readily adapted for other applications. It is also appreciated that the following description is merely illustrative of the principles of the invention, which may be applied in various ways to provide many different alternative embodiments. This description is made for the purpose of illustrating the general principles of this invention and is not meant to limit the inventive concepts in the appended claims.

An embodiment of an apparatus for coupling a prosthetic limb to a residual limb attachment system includes a prosthetic limb connector for firmly connecting the apparatus to the prosthetic limb and a residual limb connector for firmly connecting the apparatus to the residual limb attachment system. The apparatus has a physiological configuration, in which the prosthetic limb connector is axially aligned, rotationally aligned, and firmly fixed relative to the residual limb connector. The apparatus also has an overload configuration, in which the prosthetic limb is out of alignment relative to the residual limb attachment system. The apparatus transforms from the physiological configuration to the overload configuration in response to a predetermined applied load.

In an embodiment, the apparatus is adjustable to respond to a selected predetermined applied load.

In an embodiment, the apparatus automatically returns to the physiological configuration when the prosthetic limb connector is urged into alignment with the residual limb connector.

In an embodiment, the apparatus includes a bending clutch and a tension clutch. In the physiological configuration, the bending clutch firmly fixes the prosthetic limb connector in axial alignment relative to the residual limb connector, and the tension clutch firmly fixes the prosthetic limb connector in axial displacement relative to the residual limb connector, In the overload configuration, the bending clutch releases the prosthetic limb from axial alignment relative to the residual limb attachment system, and the tension clutch releases the prosthetic limb to move axially away from the residual limb attachment system.

In an embodiment, the apparatus includes a bending clutch and a torsion clutch. In the physiological configuration, the bending clutch firmly fixes the prosthetic limb connector in axial alignment relative to the residual limb connector, and the torsion clutch firmly fixes the prosthetic limb connector in rotational alignment relative to the residual limb connector. In the overload configuration, the bending clutch releases the prosthetic limb from axial alignment relative to the residual limb attachment system, and the torsion clutch releases the prosthetic limb from rotational alignment relative to the residual limb attachment system.

In an embodiment, the apparatus includes a torsion clutch and a tension clutch. In the physiological configuration, the torsion clutch firmly fixes the prosthetic limb connector in rotational alignment relative to the residual limb connector, and the tension clutch firmly fixes the prosthetic limb connector in axial displacement relative to the residual limb connector. In the overload configuration, the torsion clutch releases the prosthetic limb from rotational alignment relative to the residual limb attachment system, and the tension clutch releases the prosthetic limb to move axially away from the residual limb attachment system.

In an embodiment, each clutch acts independently to release the prosthetic limb.

In an embodiment, both clutches act together to release the prosthetic limb.

In an embodiment, the bending clutch includes a spring plunger mounted to a support structure. The support structure is coupled to a selected one of the prosthetic limb connector and the residual limb connector. The bending clutch also includes a sphere with a dimple. The sphere is coupled to a remaining one of the prosthetic limb connector and the residual limb connector. The spring plunger presses against the dimple in the physiological configuration and the spring plunger rests outside the dimple against the sphere in the overload configuration.

In an embodiment, the bending clutch includes a cantilever beam plunger mounted to a support structure. The support structure is coupled to a selected one of the prosthetic limb connector and the residual limb connector. The bending clutch also includes a sphere with a dimple. The sphere is coupled to a remaining one of the prosthetic limb connector and the residual limb connector. The cantilever beam plunger presses against the dimple in the physiological configuration and the cantilever beam plunger rests outside the dimple against the sphere in the overload configuration.

In an embodiment, the torsion clutch includes a spring plunger mounted to a support structure. The support structure is coupled to a selected one of the prosthetic limb connector and the residual limb connector. The torsion clutch also includes a socket with a dimple. The socket is coupled to a remaining one of the prosthetic limb connector and the residual limb connector. The spring plunger presses against the dimple in the physiological configuration and the spring plunger rests outside the dimple against the socket in the overload configuration.

In an embodiment, the torsion clutch includes a spring plunger mounted to a support structure. The support structure is coupled to a selected one of the prosthetic limb connector and the residual limb connector. The torsion clutch also includes a sphere with a dimple. The sphere is coupled to a remaining one of the prosthetic limb connector and the residual limb connector. The spring plunger presses against the dimple in the physiological configuration and the spring plunger rests outside the dimple against the sphere in the overload configuration.

In an embodiment, the torsion clutch includes a spring-loaded washer mounted to a support structure. A first side of the washer has alternating radial ridges and grooves. The support structure is coupled to a selected one of the prosthetic limb connector and the residual limb connector. The torsion clutch also includes a cap. A first side of the cap has alternating radial ridges and grooves. The cap is coupled to a remaining one of the prosthetic limb connector and the residual limb connector. The ridges and grooves of the washer interdigitate with the ridges and grooves of the cap in the physiological configuration and the ridges of the washer are outside the ridges of the cap in the overload configuration.

In an embodiment, the tension clutch includes a high friction interface between a selected one of the prosthetic limb connector and the residual limb connector, and a corresponding limb.

In an embodiment, the tension clutch includes a snap fit between a selected one of the prosthetic limb connector and the residual limb connector, and a corresponding limb.

In an embodiment, each clutch is independently adjustable to respond to the corresponding predetermined applied load.

In an embodiment, the bending clutch transforms from the physiological configuration to the overload configuration in response to the predetermined applied bending load, and the remaining clutch remains in the physiological configuration.

In an embodiment, the torsion clutch transforms from the physiological configuration to the overload configuration in response to the predetermined applied torsion load, and the remaining clutch remains in the physiological configuration.

In an embodiment, the tension clutch transforms from the physiological configuration to the overload configuration in response to the predetermined applied tensile load, and the remaining clutch remains in the physiological configuration.

In an embodiment, the sphere includes an external ball hex and the support structure includes a complementary straight hex socket. The external ball hex is received within the straight hex socket to rotationally couple the sphere to the support structure.

In an embodiment, the prosthetic limb connector is captive to the residual limb connector.

In an embodiment, the apparatus produces a signal upon imminent transformation from the physiological configuration to the overload configuration.

In an embodiment, the apparatus produces the signal at a pre-set load which is less than the predetermined applied load.

In an embodiment, the signal is a tactile signal.

In an embodiment, the signal is an audible signal.

In an embodiment, the apparatus includes a gage that measures loads in the apparatus in real time.

An embodiment of a method for coupling a prosthetic limb to a residual limb attachment system includes firmly connecting a prosthetic limb connector of an apparatus to the prosthetic limb, firmly connecting a residual limb connector of the apparatus to the residual limb attachment system, and transforming the apparatus from a physiological configuration to an overload configuration in response to a predetermined applied load. In the physiological configuration, the prosthetic limb connector is axially aligned, rotationally aligned, and firmly fixed relative to the residual limb connector. In the overload configuration, the prosthetic limb is out of alignment relative to the residual limb attachment system.

In an embodiment, the method includes adjusting the apparatus to respond to a selected predetermined applied load.

In an embodiment, the method includes returning the apparatus to the physiological configuration by urging the prosthetic limb into alignment with the residual connector.

An embodiment of an apparatus for coupling a prosthetic limb to a residual limb attachment system includes means for firmly connecting the apparatus to the prosthetic limb and means for firmly connecting the apparatus to the residual limb attachment system. The apparatus has a physiological configuration, in which the prosthetic limb is axially aligned, rotationally aligned, and firmly fixed relative to the residual limb. The apparatus also has an overload configuration, in which the prosthetic limb is out of alignment relative to the residual limb. The apparatus transforms from the physiological configuration to the overload configuration in response to a predetermined applied load.

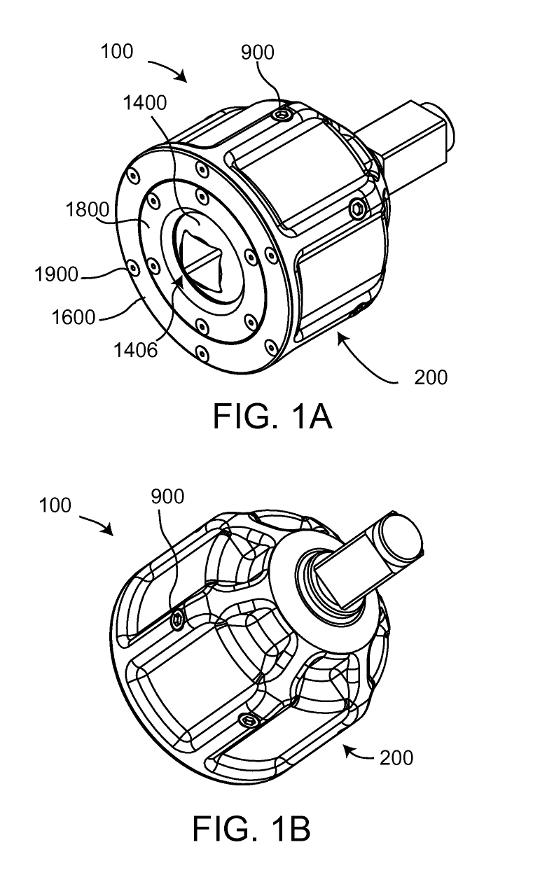

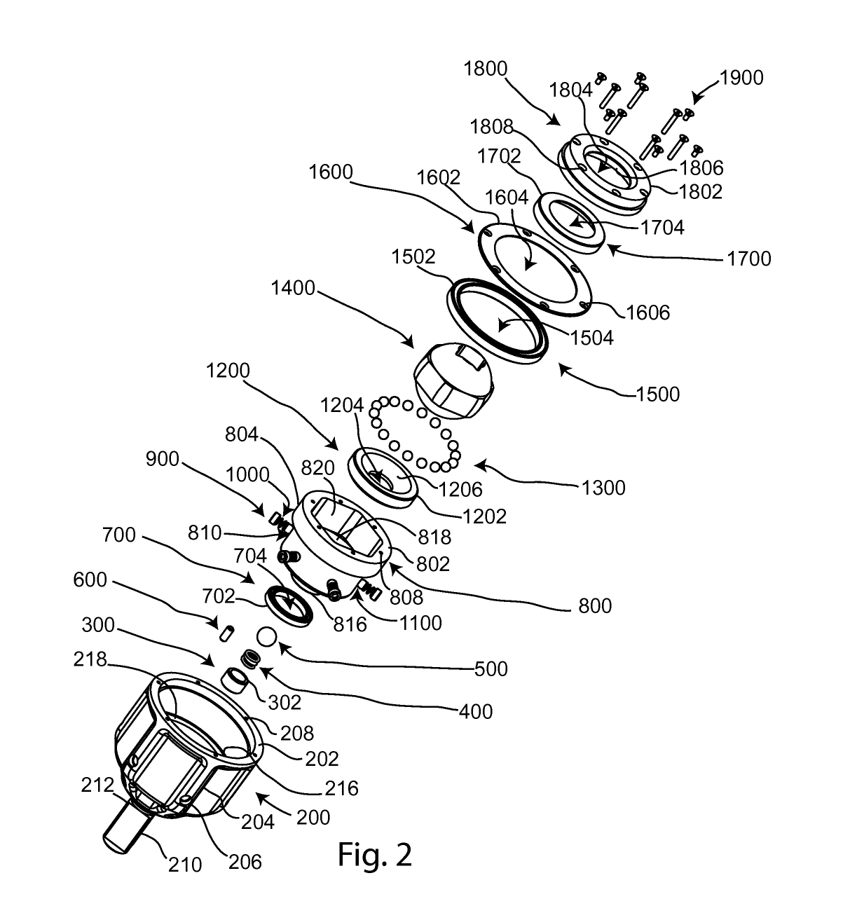

Referring to FIGS. 1A-B, 2, and 3A-B, a prosthetic connector 100 may include a housing 200, an axial cap 300, an axial spring 400, an axial ball 500, a pin 600, a bearing 700, a socket 800, a radial set screw 900, a radial spring 1000, a radial ball 1100, a bushing 1200, a ball 1300, a sphere 1400, a bearing 1500, a cap 1600, a bushing 1700, a cap 1800, and a screw 1900. A plurality of set screws 900, springs 1000, balls 1100, balls 1300, and screws 1900 may be present, as shown in the embodiment of FIG. 2.

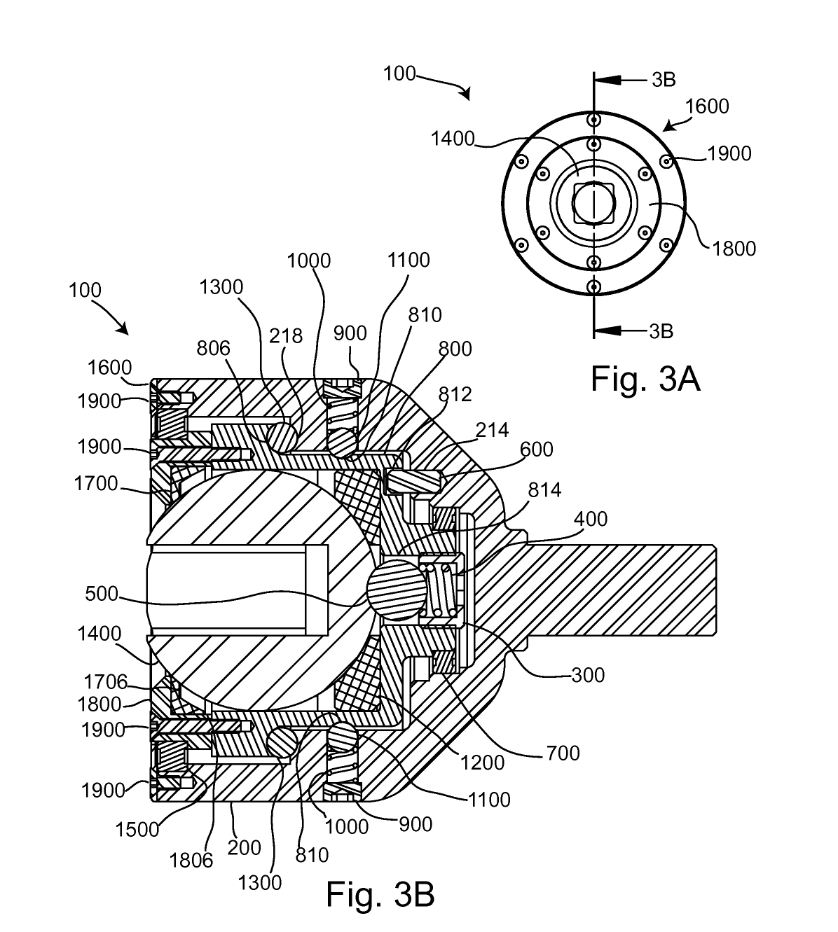

The housing 200 may form a cup 202. One or more exterior ribs 204 may project from cup 202. One or more holes 206 may be arranged around cup 202. One or more holes 208 may be arranged around the rim of cup 202. A central square shaft 210 may extend axially from the base of cup 202. A radial groove 212 may encircle shaft 210 near the base of cup 202. An eccentric hole 214 may be formed in the interior base of the cup 202, as may be seen best in FIG. 3B. The interior of cup 202 may progressively step down in diameter towards the base, so as to form at least one shelf 216 therein. A circular groove 218 may be formed on shelf 216, as may be seen best in FIG. 3B. Groove 218 may have an arcuate cross section and may extend around cup 202.

The axial cap 300 may form a cup 302. Cap 300 may be externally threaded.

The bearing 700 may form a ring 702 with a central aperture 704.

The socket 800 may form a cup 802. A flange 804 may extend around the rim of cup 802. A circular groove 806 may be formed in an axial face of flange 804, as may be seen best in FIG. 3B. Groove 806 may have an arcuate cross section and may extend around cup 802. One or more holes 808 may be arranged around the rim of cup 802. One or more exterior dimples 810 may be arranged around cup 802. An eccentric arcuate groove 812 may be formed in the exterior base of the cup 802, as may be seen best in FIG. 3B. A central hole 814 may be formed through the base of the cup 802, as may be seen best in FIG. 3B. Hole 814 may extend into or through a boss 816 that projects externally from the base of the cup 802. Hole 814 may be internally threaded. A counterbore 818 may be formed inside the base of the cup 802 around hole 814. A hex socket 820 may be formed inside cup 802 around counterbore 818.

The bushing 1200 may form a ring 1202 with a central aperture 1204. The ring 1202 may have a concave spherical surface 1206 surrounding the aperture 1204. Bushing 1200 may be fabricated from plastic, polymer, elastomer, rubber, or other lubricious or compliant material.

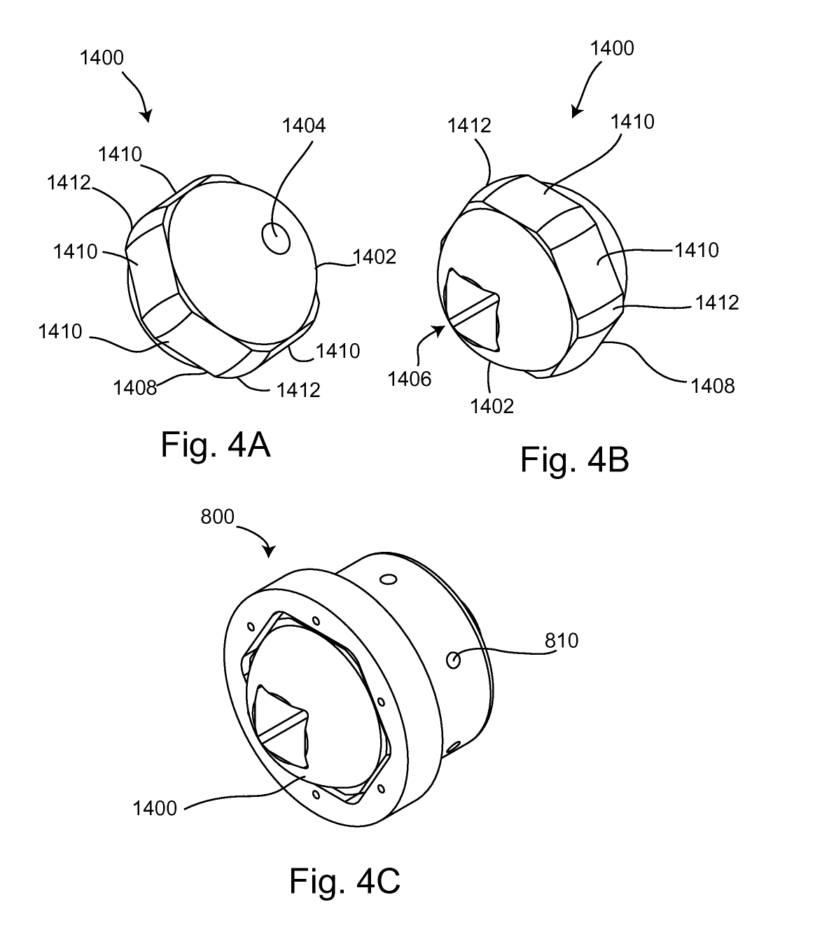

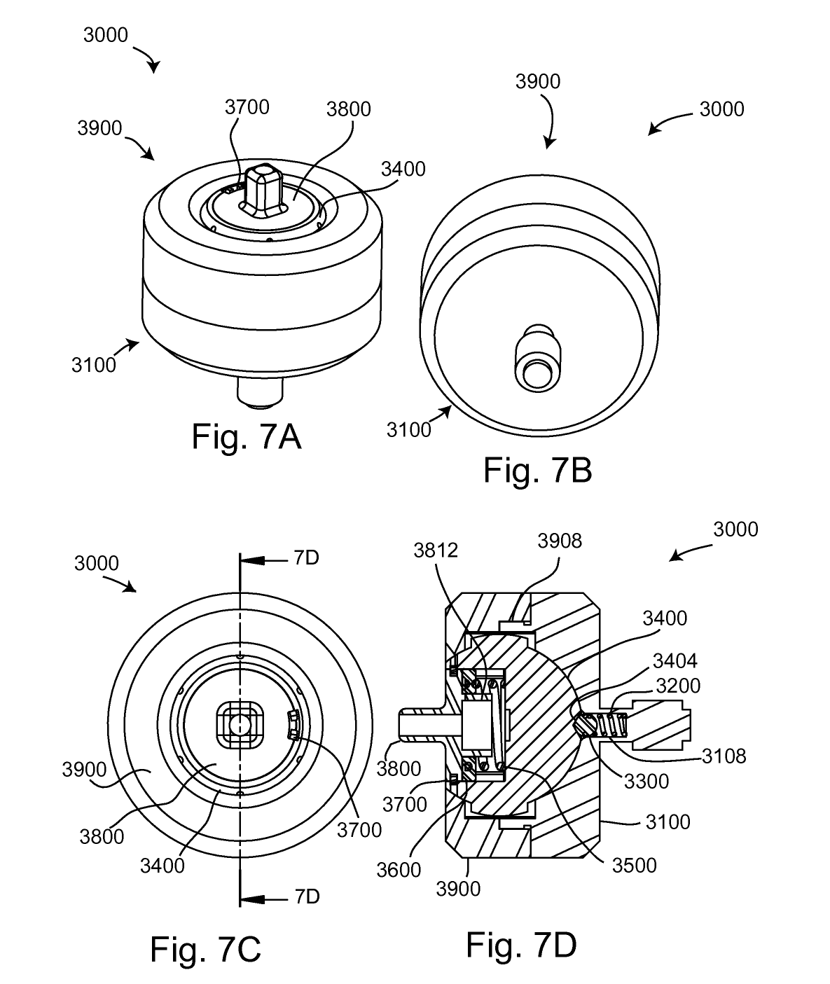

Referring now to FIGS. 4A-4B, the sphere 1400 has a convex spherical surface 1402 which may include a dimple 1404, a square socket 1406 opposite the dimple 1404, and an equatorial external hex 1408 located between the dimple 1404 and the socket 1406. The hex 1408 may have convex rounded sides 1410 and corners 1412 so as to form a ball hex. With reference to FIG. 4C, it can be appreciated that convex rounded hex 1408 fits within hex socket 820 so as to couple sphere 1400 and socket 800 in axial rotation, while permitting sphere 1400 to spherically pivot relative to socket 800. More specifically, sphere 1400 is prevented from rotating relative to socket 800 about a center longitudinal axis of socket 800, but is permitted to pivot relative to socket 800 in other directions. FIG. 7D may further clarify the concept by showing a cross section through similar sphere and socket components, taken through a more pronounced portion of the corresponding hex features.

Returning to FIGS. 1A-B, 2, and 3A-B, the bearing 1500 may form a ring 1502 with a central aperture 1504.

The cap 1600 may form a ring 1602 with a central aperture 1604. One or more holes 1606 may be arranged around the ring 1602.

The bushing 1700 may form a ring 1702 with a central aperture 1704. The ring 1702 may have a concave side 1706 surrounding the aperture 1704, as may be seen best in FIG. 3B. Bushing 1700 may be fabricated from plastic, polymer, elastomer, rubber, or other lubricious or compliant material.

The cap 1800 may form a ring 1802 with a central aperture 1804. The ring 1802 may have a counterbore 1806 surrounding the aperture 1804, as may be seen best in FIG. 3B. One or more holes 1808 may be arranged around the ring 1802.

FIG. 3B shows a cross section through a fully assembled prosthetic connector 100. With reference to FIGS. 2 and 3B, sphere 1400 is sandwiched between bushings 1200 and 1700, with concave spherical surface 1206 and concave side 1706 facing the convex spherical surface 1402 so that dimple 1404 is exposed through aperture 1204 and socket 1406 is exposed through aperture 1704. Ball 500 is pressed into dimple 1404 of sphere 1400 by spring 400, which is supported by axial cap 300. Cap 300 may be described as a support structure for spring 400 and ball 500. Sphere 1400, bushings 1200 and 1700, ball 500, spring 400, and cap 300 are received within socket 800 so that the hex 1408 rests within the hex socket 820, as may be seen best in FIG. 4C. Axial cap 300 is fixed within hole 814 of socket 800; for example, axial cap 300 may be threaded into hole 814 so that cap 300 is supported by housing 800. Housing 800 may also be described as a support structure for cap 300. The force pressing ball 500 against sphere 1400 is proportional to the insertion depth of cap 300 into hole 814, and may be adjusted at any time in this embodiment by partially disassembling the prosthetic connector 100 to expose axial cap 300. Aperture 704 of bearing 700 is pressed over boss 816. Cap 1800 is fastened to the rim of cup 802 with one or more screws 1900 so that bushing 1700 is received in counterbore 1806 and socket 1406 is exposed through aperture 1804. Screw 1900 extends through hole 1808 and threads into hole 808. Aperture 1504 of bearing 1500 is pressed over the outer diameter of ring 1802.

Pin 600 is received by hole 214 in housing 200. One or more balls 1300 are received by groove 218; the arcuate cross section of groove 218 may complement the radius of ball 1300. Cap 300, spring 400, ball 500, bearing 700, socket 800, bushing 1200, sphere 1400, bearing 1500, bushing 1700, cap 1800, and related screw or screws 1900 slide within cup 202 so that groove 812 receives a protruding portion of pin 600 and groove 806 rests against ball or balls 1300. The outer diameters of bearings 700 and 1500 engage complementary diameters within cup 200 so that socket 800 and attached components are rotatably supported within housing 200 within rotational limits set by the action of pin 600 in groove 812. Cap 1600 is fastened to the rim of cup 200 with one or more screws 1900 so that socket 1406 is exposed through aperture 1604. Screw 1900 extends through hole 1606 and threads into hole 208.

Each hole 206 receives a ball 1100, followed by a spring 1000, and finally a set screw 900 so that ball 1100 is pressed against socket 800 by spring 1000 as set screw 900 is threaded into hole 206. The force pressing ball 1100 against socket 800 is proportional to the insertion depth of set screw 900 in hole 206, and may be adjusted at any time in this embodiment. Once all of the balls 1100, springs 1000, and set screws 900 are installed, socket 800 may be axially rotated to a position in which each ball 1100 engages a corresponding dimple 810.

The socket 1406 of the sphere may rotationally and axially couple the sphere 1406 to a residual limb attachment system, such as a direct skeletal attachment system (not shown) or a socket type attachment system (not shown), by sliding onto a correspondingly-shaped pin, post, or peg of the attachment system. In this situation, the socket 1406 may be described as a residual limb connector. The fit between the socket 1406 and peg may be so snug that friction alone provides adequate axial retention force; alternatively, the peg and/or socket may include corresponding retention features (not shown) to releasably secure the components together. Friction or other axial retention elements may also be described as tension clutches. An alternate version of sphere 1400 may include a positive residual limb connector, such as a shaft, instead of a negative feature, such as a socket. Other alternate versions of sphere 1400 may couple to a prosthetic limb instead of a direct skeletal attachment system, in which case the socket or shaft may be described as a prosthetic limb connector.

The ribs 204 and square shaft 210 of housing 200 may rotationally couple the housing 200 to a prosthetic limb (not shown) by sliding into a complementary socket of the prosthetic limb. The interface between the housing 200 and the socket of the prosthetic limb may be so snug that the high friction alone provides an axial retention force, which may also be described as a tension clutch; alternatively, the housing and/or socket may include corresponding retention features to releasably axially secure the components together. These retention features may be considered alternate embodiments of tension clutches. In one example, the groove 212 may axially couple the housing 200 to the prosthetic limb by receiving a corresponding ball plunger, retaining ring, or snap ring. It can be appreciated that any of these retention components may provide a snap fit interconnection. Other releasable retaining components may also be used. An alternate version of housing 200 may include a negative prosthetic connector, such as a socket, instead of a positive feature, such as external ribs or a shaft. Other alternate versions of housing 200 may couple to a residual limb attachment system instead of a prosthetic limb, in which case the socket or shaft may be described as a residual limb connector.

The fully assembled prosthetic connector 100 provides a rigid, stable connection between a remaining limb and a prosthetic limb at loads below a predetermined threshold. Sphere 1400 is held in a fixed position relative to housing 200 by the remaining components in the assembly. Ball 500 in dimple 1404 holds sphere 1400 axially stable relative to housing 200. Ball 1100 in dimple 810 holds sphere 1400 rotationally stable relative to housing 200 due to the engagement of hex 1408 in hex socket 820. Thus, prosthetic connector 100 holds socket 1406 axially aligned, rotationally aligned, and firmly fixed relative to shaft 210. This condition may be described as a physiological configuration, since it is a condition in which the prosthetic limb is functionally oriented relative to the residual limb so as to replicate aspects of the amputated limb. The stabilizing effect of ball 500 and ball 1100 may be adjusted by increasing or decreasing the insertion depth of cap 300 in hole 814 or set screw 900 in hole 206, respectively. The axial and rotational stabilizing effects may be independently adjusted.

The fully assembled prosthetic connector 100 also provides a safety release mechanism that is adapted to give way, or release, in response to an excess mechanical load, thereby preventing transmission of the excess load to the residual limb, its soft tissue, or components of the direct skeletal attachment system. In one example, an excess bending load against a prosthetic limb will cause housing 200 to rotate relative to sphere 1400 so that ball 500 rides up and out of dimple 1404, thereby releasing the axial stabilizing effect of the prosthetic connector 100. Ball 500, spring 400, and cap 300 form a bending clutch, or detent, with sphere 1400 which permits shaft 210 to go out of axial alignment relative to socket 1406. This condition may be described as an overload configuration. Bending clutches which rely upon a single axial ball plunger may provide consistent response to bending loads applied from any angle. In another example, an excess torsional load on the prosthetic limb will cause housing 200 to rotate relative to sphere 1400 in socket 800 so that ball or balls 1100 ride up and out of dimple or dimples 810, thereby releasing the rotational stabilizing effect of the prosthetic connector 100. Ball 1100, spring 1000, and set screw 900 form a torsion clutch, or detent with sphere 1400 which permits shaft 210 to go out of torsional alignment with socket 1406. This condition may also be described as an overload configuration. In yet another example, an excess tensile force along the prosthetic limb will cause housing 200 to separate from the prosthetic limb, or sphere 1400 to separate, or axially displace away, from the direct skeletal attachment system. This condition may also be described as an overload configuration. Furthermore, under combined loading, prosthetic connector 100 may exhibit two or more independent overload configurations simultaneously.

The safety release mechanism provided by prosthetic connector 100 is readily resettable after responding to an excess load. In the case of bending or rotational overload, the prosthetic limb is merely repositioned in its normal physiologic orientation relative to the residual limb attachment system, and balls 500 and/or 1100 automatically snap into position in their respective dimples 1404, 810 to return the prosthetic connector 100 to the physiological configuration. In the case of tensile overload, the prosthetic limb is reattached to the prosthetic connector 100, or the prosthetic connector 100 is reattached to the direct skeletal attachment system.

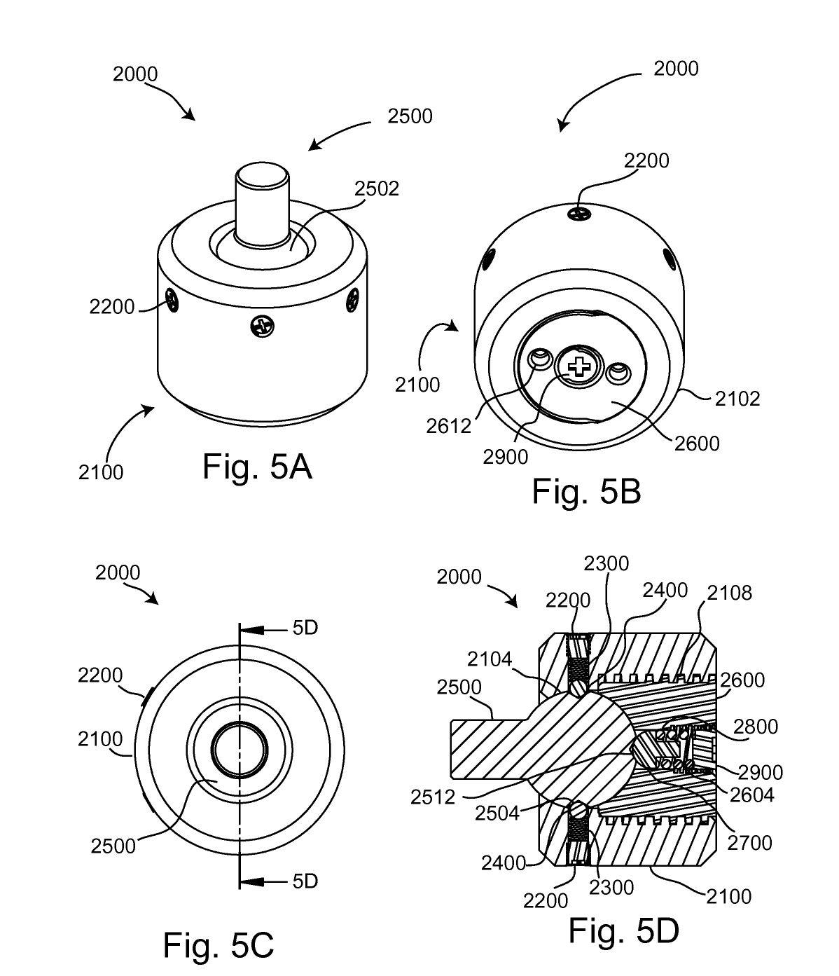

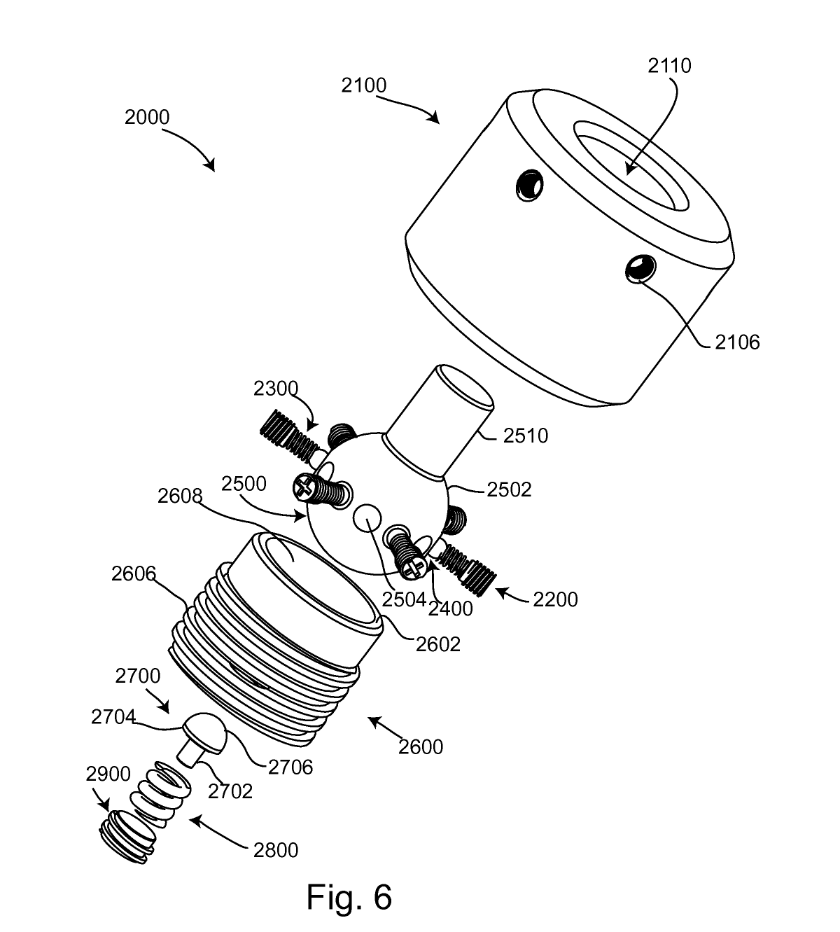

Referring now to FIGS. 5A-5D and 6, a prosthetic connector 2000 may include a housing 2100, a set screw 2200, a spring 2300, a ball 2400, a sphere 2500, a socket 2600, a plunger 2700, a spring 2800, and a cap 2900.

The housing 2100 may form a cup 2102. One or more holes 2106 may be arranged around cup 2102. The interior base of the cup 2102 may have a concave spherical surface 2104 surrounding an aperture 2110. Cup 2102 may have internal threads 2108.

The sphere 2500 may have a convex spherical surface 2502. A shaft 2510 may extend from one side of the sphere 2500. Surface 2502 may have a dimple 2512 directly opposite the shaft 2510. Surface 2502 may also have one or more dimples 2504 arranged around sphere 2500 between shaft 2510 and dimple 2512. In this embodiment, dimples 2504 and 2512 are conical depressions; spherical, cylindrical, elliptical, spot face, planar, or other shapes could also be employed in this or any other embodiment.

The socket 2600 may form a tube 2602 with internal and external threads 2604, 2606. The tube 2602 may have a concave spherical surface 2608 in one end around the internal threads 2604. Socket 2600 may also have eccentric holes 2612 opposite the spherical surface 2608.

The plunger 2700 may have a shaft 2702 with an enlarged head 2704. Head 2704 may have a convex spherical surface 2706 opposite the shaft 2702.

FIG. 5D shows a cross section view of the fully assembled prosthetic connector 2000. With reference to FIGS. 5D and 6, convex spherical surface 2502 is sandwiched between concave spherical surfaces 2104 and 2608, with shaft 2510 protruding through aperture 2110. Socket 2600 is threaded into housing 2100. Cap 2900 is threaded into tube 2602 so that spring 2800 and plunger 2700 are sandwiched between cap 2900 and sphere 2500, with shaft 2702 received within spring 2800 and convex spherical surface 2706 nestled in dimple 2504. Cap 2900 may be described as a support structure for spring 2800 and plunger 2700. Socket 2600 may be described as a support structure for cap 2900. Each hole 2106 receives a ball 2400, which is pressed against spherical surface 2502 by spring 2300 as set screw 2200 is threaded into hole 2106.

Shaft 2510 may be adapted to be firmly connected within a socket of a residual limb attachment system, such as a direct skeletal attachment system or socket type attachment system. In this case, shaft 2510 may be described as a residual limb connector. In one example, a longitudinal flat may be added to shaft 2510 for rotational control and a circumferential groove may be added for axial retention. Housing 2100 may be adapted to be firmly connected within a socket of a prosthetic limb by adding ribs similar to those depicted for prosthetic connector 100, and one or more dimples for axial retention. In this case, housing 2100 may be described as a prosthetic limb connector. Other variations are contemplated, in which sphere 2500 and/or housing 2100 includes a socket or other internal feature rather than an external feature. Furthermore, prosthetic connector 2000 may be reversed so that housing 2100 connects to a direct skeletal attachment system and sphere 2500 connects to a prosthetic limb.

Prosthetic connector 2000 provides a safety release mechanism that is adapted to release, or give way, in response to an excess bending or torsional load. An excess bending load against an attached prosthetic limb will cause housing 2100 to rotate relative to sphere 2500 so that plunger 2700 rides up and out of dimple 2512. Plunger 2700, spring 2800, and set screw 2900 form a bending clutch with sphere 2500 which permits housing 2100 to go out of axial alignment with shaft 2510. At the same time, ball 2400 will also ride up and out of dimple 2504. Ball 2400, spring 2300, and set screw 2200 form a torsion clutch with sphere 2500 which permits housing 2100 to go out of torsional alignment with shaft 2510. Thus, prosthetic connector 2000 releases in bending and torsion in response to a bending overload. However, an excess torsional load against the prosthetic limb will cause torque release without necessarily triggering bending release. Furthermore, the prosthetic limb could rotate until balls 2400 engage dimples 2504, and then automatically stabilize itself in the rotated position.

Referring now to FIGS. 7A-D and 8, a prosthetic connector 3000 may include a housing 3100, a spring 3200, a ball 3300, a sphere 3400, a spring 3500, a washer 3600, a retaining ring 3700, a cap 3800, and a cap 3900.

The housing 3100 may form a circular disk 3102 with a central shaft 3104 protruding from one side. The disk may have a hex socket 3106 formed opposite the shaft 3104. A central blind hole 3108 may be formed in the base of the hex socket 3106, and may extend into the shaft 3104. External threads 3110 may be provided around hex socket 3106. A concave spherical surface 3112 may be formed in the base of the hex socket 3106 around the hole 3108.

The sphere 3400 has a convex spherical surface 3402 which may include a dimple 3404, a socket 3406 opposite the dimple 3404, and an equatorial external hex 3408 located between the dimple 3404 and the socket 3406. The hex 3408 may have convex rounded sides 3410 and corners 3412 so as to form a ball hex. It can be appreciated that hex 3408 fits within hex socket 3106 so as to rotationally couple sphere 3400 and housing 3100, while permitting sphere 3400 to pivot relative to housing 3100. A groove 3414 may be formed around the mouth of socket 3406, for example, a groove for a retaining ring. Socket 3406 may be noncircular in cross section; for example, socket 3406 may have outwardly projecting lobes 3416 which give socket 3406 a somewhat square cross sectional shape. Sphere 3400 may also include one or more holes 3418 arranged around the rim of socket 3406.

The washer 3600 may form a ring 3602 with a central aperture 3604. Ring 3602 may have enlarged peripheral tabs 3606 which project from the nominal outer diameter of ring 3602. One side of ring 3602 may have alternating radial grooves 3608 and ridges 3610 so as to form an undulating surface.

The cap 3800 may form a circular disk 3802 with a central square shaft 3804 protruding from one side. A groove 3806 may be formed around the outer diameter of the disk 3802. A boss 3808 may protrude from the disk 3802 opposite the shaft 3804. A central hole 3810 may extend through the boss 3808, disk 3802, and shaft 3804. A counterbore 3812 may be formed in the boss 3808 around the hole 3810. One or more ridges 3814 may project from the disk 3802 around the boss 3808.

The cap 3900 may form a ring 3902 with a central aperture 3904. The ring 3902 may have a hex socket 3906 on one side surrounding the aperture 3904. A concave spherical surface 3910 may be formed in the base of the socket 3906 around the aperture 3904. Internal threads 3908 may be formed around the mouth of the hex socket 3906.

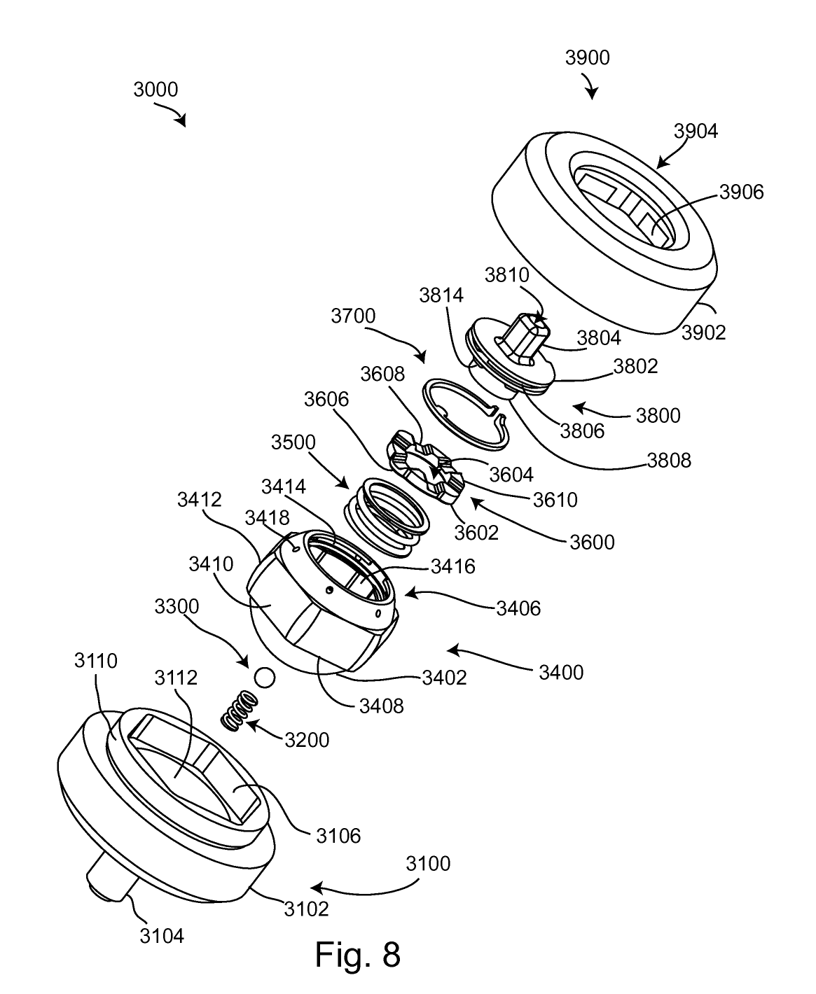

FIG. 7D shows a cross section view of the fully assembled prosthetic connector 3000. With reference to FIGS. 7D and 8, convex spherical surface 3402 is sandwiched between concave spherical surfaces 3112 and 3910, with socket 3406 exposed through aperture 3904 and hex 3408 received within hex sockets 3106 and 3906. Spring 3200 and ball 3300 are received within hole 3108 so that ball 3300 is pressed into dimple 3404 by spring 3200. Housing 3100 may be described as a support structure for spring 3200 and ball 3300. Housing 3100 and cap 3900 are secured together with threads 3110, 3908. Spring 3500 and washer 3600 are received within socket 3406 and covered by cap 3800 so that ridges 3610 interdigitate with ridges 3814 as spring 3500 presses washer 3600 against cap 3800. Tabs 3606 engage lobes 3416 to torsionally couple washer 3600 to sphere 3400.

Prosthetic connector 3000 provides a safety release mechanism that is adapted to release in response to an excess bending or torsional load. An excess bending load against an attached prosthetic limb will cause housing 3100 to rotate relative to sphere 3400 so that ball 3300 rides up and out of dimple 3404. Ball 3300, spring 3200, and the bottom of hole 3108 in housing 3100 form a bending clutch with sphere 3400 which permits shaft 3104 to go out of axial alignment relative to shaft 3804. An excess torsional load on the prosthetic limb will cause cap 3800 to rotate relative to sphere 3400 so that ridges 3610 ride up and over ridges 3814. Washer 3600, spring 3500, and socket 3400 form a torsion clutch with cap 3800 which permits shaft 3104 to go out of torsional alignment relative to shaft 3804. Furthermore, the prosthetic limb could rotate until ridges and grooves 3608, 3610 re-engage ridges 3814, and then automatically stabilize itself in the rotated position. Prosthetic connector 3000 provides independent bending and torque release, and is readily resettable after release.

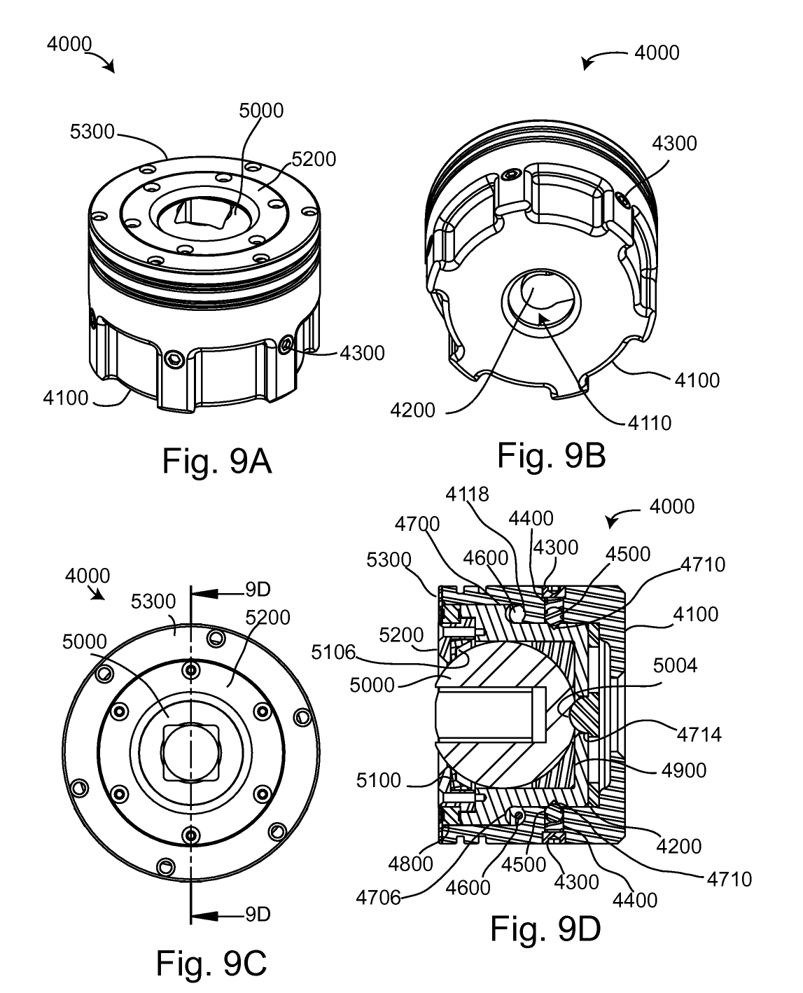

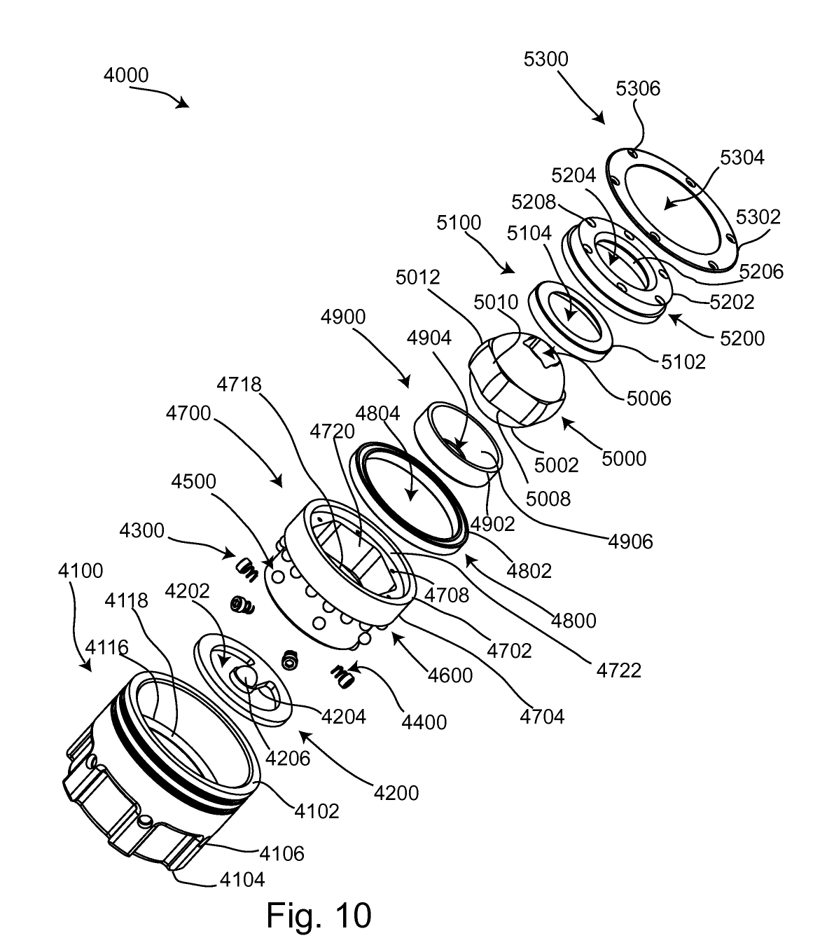

Referring now to FIGS. 9A-9D and 10, a prosthetic connector 4000 may include a housing 4100, a ring 4200, a set screw 4300, a spring 4400, a ball 4500, a ball 4600, a socket 4700, a bearing 4800, a bushing 4900, a sphere 5000, a bushing 5100, a cap 5200, and a cap 5300. A plurality of set screws 4300, springs 4400, balls 4500, and balls 4600 may be present. Fasteners, such as screw 1900, may also be present.

The housing 4100 may form a cup 4102. One or more exterior ribs 4104 may project from cup 4102. One or more holes 4106 may be arranged around cup 4102. One or more holes (not shown) may be arranged around the rim of cup 4102, similar to hole 208 of housing 200. The base of cup 4102 may have a central aperture 4110. The interior of cup 4102 may progressively step down in diameter towards the base, so as to form at least one shelf 4116 therein. A circular groove 4118 may be formed on shelf 4116. Groove 4118 may have an arcuate cross section and may extend around cup 4102.

The ring 4200 may include a central aperture 4202. A cantilever beam 4204 may project from a peripheral portion of ring 4200 into the aperture 4202, and may terminate in a domed head 4206 positioned at or near the center of ring 4200. Cantilever beam 4204 and head 4206 form a cantilever beam plunger supported by the periphery of ring 4200.

The socket 4700 may form a cup 4702. A flange 4704 may extend around the rim of cup 4702. A circular groove 4706 may be formed in an axial face of flange 4704, as may be seen best in FIG. 9D. Groove 4706 may have an arcuate cross section and may extend around cup 4702. One or more holes 4708 may be arranged around cup 4702 proximate the rim. One or more exterior dimples 4710 may be arranged around cup 4702, as may be seen best in FIG. 9D. A central hole 4714 may be formed through the base of the cup 4702, as may be seen best in FIG. 9D. A counterbore 4718 may be formed inside the base of the cup 4702 around hole 4714. A hex socket 4720 may be formed inside cup 4702 around counterbore 4718. A counterbore 4722 may be formed inside the rim of cup 4702 around socket 4702. Holes 4708 may be located in counterbore 4722, as shown.

The bearing 4800 may form a ring 4802 with a central aperture 4804. Bearing 4800 may resemble, or be identical to, bearing 1500.

The bushing 4900 may form a ring 4902 with a central aperture 4904. The ring 4902 may have a concave spherical surface 4906 surrounding the aperture 4904. Bushing 4900 may resemble, or be identical to, bushing 1200. Bushing 4900 may be fabricated from plastic, polymer, elastomer, rubber, or other lubricious or compliant material.

The sphere 5000 has a convex spherical surface which may be interrupted by a dimple 5004, a square socket 5006 opposite the dimple 5004, and a generally equatorial external hex 5008 located between the dimple 5004 and the socket 5006. The hex 5008 may have convex rounded sides 5010 and corners 5012 to form a ball hex. Sphere 5000 may resemble, or be identical to, sphere 1400.

The bushing 5100 may form a ring 5102 with a central aperture 5104. The ring 5102 may have a concave side 5106 surrounding the aperture 5104, as may be seen best in FIG. 9D. Bushing 5100 may resemble, or be identical to, bushing 1700. Bushing 5100 may be fabricated from plastic, polymer, elastomer, rubber, or other lubricious or compliant material.

The cap 5200 may form a ring 5202 with a central aperture 5204. The ring 5202 may have a counterbore 5206 surrounding the aperture 5204, as may be seen best in FIG. 9D. One or more holes 5208 may be arranged around the ring 5202. Cap 5200 may resemble, or be identical to, cap 1800.

The cap 5300 may form a ring 5302 with a central aperture 5304. One or more holes 5306 may be arranged around the ring 5302. Cap 5300 may resemble, or be identical to, cap 1600.

FIG. 9D shows a cross section view of the fully assembled prosthetic connector 4000. With reference to FIGS. 9D and 10, convex spherical surface 5002 is sandwiched between concave spherical surface 4906 and concave side 5106, so that dimple 5004 is exposed through hole 4714 and socket 5006 is exposed through aperture 5104. Sphere 5000, bushing 4900, and bushing 5100 are received within cup 4702 so that hex 5006 rests within hex socket 4720. Cap 5200 is fastened to the rim of cup 4702 by one or more screws 1900 so that bushing 5100 is received in counterbore 5206 and socket 5006 is exposed through aperture 5204. Screw 1900 extends through hole 5208 and threads into hole 4708. Aperture 4804 of bearing 4800 is pressed over the outer diameter of ring 5202.

Ring 4200 is received inside the base of cup 4102. Housing 4100 may be described as a support structure for ring 4200. One or more balls 4600 are received by groove 4706; the arcuate cross section of groove 4706 may complement the radius of ball 4600. Socket 4700, bushing 4900, sphere 5000, bushing 5100, cap 5200, bearing 4800, and screws 1900 slide within cup 4102 so that groove 4706 rests against ball or balls 4600 and head 4206 is pressed into dimple 5004 by cantilever beam deflection of beam 4204. The outer diameter of bearing 4800 engages a complementary diameter within cup 4102 so that socket 4700 and attached components rotate within housing 4100. Cap 5300 is fastened to the rim of cup 4102 with one or more screws 1900 (not shown) so that socket 5006 is exposed through aperture 5304. Screw 1900 extends through hole 5306 and threads into a hole (not shown) in the rim of cup 4102.

Each hole 4106 receives a ball 4500, spring 4400, and set screw 4300 so that ball 4500 is pressed against socket 4700 by spring 4400 as set screw 4300 is threaded into hole 4106. Once all of the balls 4500, springs 4400, and set screws 4300 have been installed, socket 4700 may be axially rotated to a position in which each ball 4500 engages a corresponding dimple 4710.

The socket 5006 of the sphere 5000 may rotationally and axially couple the sphere 5006 to a socket-type or direct-skeletal attachment system (not shown) by sliding onto a correspondingly-shaped pin, post, or peg of the attachment system. In this situation, socket 5006 may be described as a residual limb connector. The fit between the socket 5006 and peg may be so snug that friction alone provides adequate axial retention force; alternatively, the peg and/or socket may include corresponding retention features to releasably secure the components together. An alternate version of sphere 5000 may have a positive residual limb connector, such as a post, peg, or pin instead of an internal feature such as a socket. Additional alternate embodiments of sphere 5000 may be coupled to a prosthetic limb, in which case the socket or shaft may be described as a prosthetic connector.

The ribs 4104 of housing 4100 may rotationally and axially couple the housing 4100 to a prosthetic limb (not shown) by sliding into a correspondingly-shaped socket of the prosthetic limb. Thus, the ribs 4104 may be described as a prosthetic connector. The fit between the housing 4100 and the socket of the prosthetic limb may be so snug that friction alone provides adequate retention force; alternatively, the housing and/or socket may include corresponding retention features to releasably secure the components together. An alternate version of housing 4100 may include a recessed or internal prosthetic connector, such as a socket or grooves, instead of a positive feature like ribs or a shaft. Other alternate versions of housing 4100 may couple to a residual limb attachment system instead of a prosthetic limb, in which case the socket or shaft may be described as a residual limb connector.

The fully assembled prosthetic connector 4000 provides a safety release mechanism that is adapted to release, or slip, in response to an excess bending or torsional load. For example, an excess bending load against a prosthetic limb will cause housing 4100 to rotate relative to sphere 5000 so that head 4206 rides up and out of dimple 5004. Ring 4200 and housing 4100 form a bending clutch with sphere 5000 which permits housing 4100 to go out of axial alignment with socket 5006. In another example, an excess torsional load on the prosthetic limb will cause housing 4100 to rotate relative to sphere 5000 in socket 4700 so that each ball 4500 rides up out of each corresponding dimple 4710. Ball 4500, spring 4400, and set screw 4300 form a torsion clutch with socket 4700 which permits housing 4100 to go out of rotational alignment relative to socket 5006. Prosthetic connector 4000 provides independent bending and torque release.

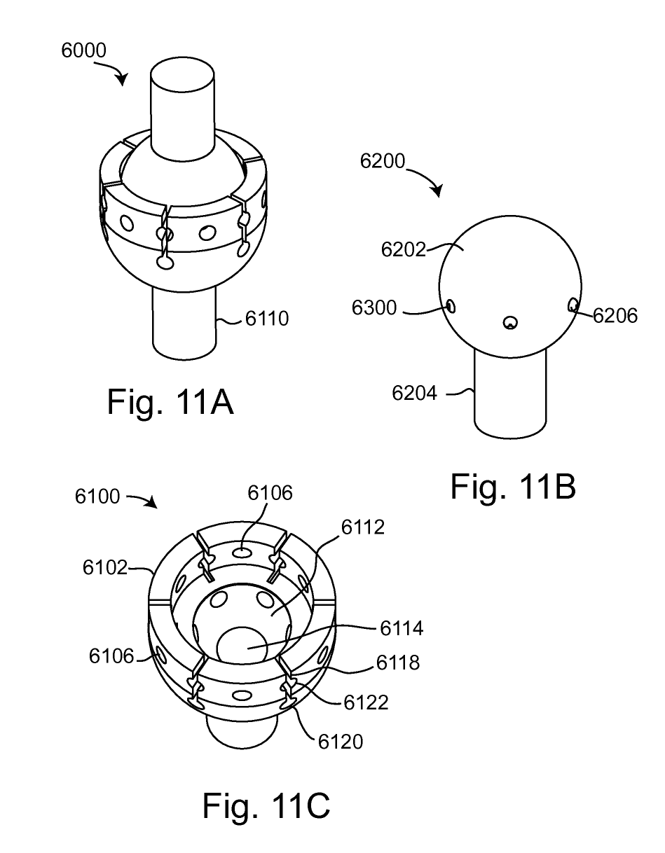

Referring now to FIGS. 11A-11C, a prosthetic connector 6000 may include a housing 6100, a sphere 6200, and a ball plunger 6300. More than one ball plunger 6300 may be included.

The housing 6100 may form a cup 6102. One or more holes 6106 may be arranged around cup 6102. One or more through holes 6108 may be arranged around the exterior of cup 6102. A central shaft 6110 may extend axially from the base of cup 6102. The interior base of cup 6102 may have a concave spherical surface 6112, or may be flat or tapered. The center interior base of cup 6102 may include a spot face 6114. Cup 6102 may be split into a plurality of segments 6116 by a slot 6118 that extends from the rim toward the base, so that cup 6102 resembles a collet. More than one slot 6118 may be included. Slot 6118 may terminate at or near a hole 6120. Slot 6118 may include another hole 6122 between the rim and hole 6120.

The sphere 6200 has a convex spherical surface 6202. A shaft 6204 may protrude from the sphere 6200. The spherical surface 6202 may be interrupted by one or more holes 6206, which may be oriented perpendicular to the shaft 6204.

The ball plunger 6300 may be a pre-assembled unit that includes a ball, spring, and set screw similar to, or identical to, the ball, screw, and set screw units described elsewhere.

A ball plunger 6300 is inserted, such as by threading, into each hole 6206 so that at least a portion of the ball protrudes from the spherical surface 6202. Spherical surface 6202 with assembled ball plungers 6300 is received within cup 6102, which may be able to expand like a collet to capture the spherical surface 6202 within the housing 6100. Ball plungers 6300 spring outwardly to press into holes 6106 or 6122 when shaft 6204 is axially aligned with shaft 6110 and sphere 6200 is rotated to position the balls in the holes 6106 or 6122.

Shaft 6110 may be adapted to be firmly connected within a socket of a residual limb attachment system, such as a direct skeletal attachment system or socket-type attachment system. In this case, shaft 6110 may be described as a residual limb connector. In one example, a longitudinal flat may be added to shaft 6110 for rotational control and a circumferential groove may be added for axial retention. Shaft 6110 may also be replaced by an internal feature, such as a square socket, for connection to a positive feature on the residual limb attachment system. Shaft 6110, or its internal equivalent, may also be adapted to be firmly connected to a prosthetic limb. In this case, shaft 6110 may be described as a prosthetic limb connector. Similarly, shaft 6204 may serve as a residual limb connector or a prosthetic limb connector, and may be replaced by an internal feature.

Prosthetic connector 6000 provides a safety release mechanism that is adapted to release, or give way, in response to an excess bending or torsional load. An excess bending load against an attached prosthetic limb will cause housing 6100 to rotate relative to sphere 6200 so that ball plunger 6300 rides up and out of hole 6106 or hole 6122. Ball plunger 6300 forms a bending clutch with housing 6100 which permits housing 6100 to go out of axial alignment with shaft 6204. Ball plunger also forms a torsion clutch with housing 6100 which permits housing 6100 to go out of torsional alignment with shaft 6204. Thus, prosthetic connector 6000 releases in both bending and torsion in response to a bending or torsion overload. Furthermore, the prosthetic limb could rotate until ball plungers 2300 engage holes 6106 or holes 6122, and then automatically stabilize in the rotated position.

The components of the prosthetic connectors disclosed herein may be fabricated of metal, polymer, ceramic, glass, rubber, elastomer, or other suitable material. Each component may be fabricated from a different material. Any component may include a combination of materials.

Any of the components disclosed herein may include surface treatments or additives in one or more of the component materials to provide beneficial effects such as anti-microbial, analgesic or anti-inflammatory properties. Any of the components disclosed herein may include coatings or treatments to provide surface roughening, including but not limited to knurling or porous coating, among others. Such treatments may be directionally applied to promote movement between component parts in one direction, and/or increase friction between component parts in another direction.

While the foregoing discussion has described various bending, torsional, or tensile loads against which the prosthetic connector 100 and other embodiments may provide protection, it is appreciated that combined loading is more often the case. The prosthetic connector 100 and other embodiments disclosed herein provide, or may be readily adapted to provide, safety overload protection for combined loading. If and when applied forces exceed a predetermined safe limit in any direction, or in a combination of directions, the prosthetic connector responds by releasing in one or more directions.

Various other detent embodiments are contemplated. In one example, detents may act directly against hex 1408 to provide torque release. In another example, a prosthetic connector may incorporate piezoelectric elements which, under stress, electrically trigger a safety release mechanism. In yet another example, a prosthetic connector may be stabilized with magnetic detents, which may be permanent magnets or electromagnets. Electromagnets could be controlled via piezoelectric elements. In yet another example, a prosthetic connector is contemplated in which the springs disclosed above are replaced with hydraulic elements. In yet another example, a modified prosthetic connector similar to connector 2000 may have a flattened plunger which presses against a flattened end portion of a sphere component. This version may be even more easily reset than other versions due to the potentially large flat contact area between the plunger and sphere.

Each of the detent mechanisms disclosed above may be adjusted in order to tailor the overload threshold to a particular patient's needs. A patient with osteoporotic bone or a brand new direct skeletal attachment system may require more protection, hence a lower overload threshold, while an athletic patient with a firmly incorporated direct skeletal attachment system may tolerate, or even enjoy, a higher overload threshold. The ball-spring-set-screw mechanisms may be adjusted by increasing or decreasing the depth of the set screw in its corresponding hole. A certain amount of disassembly may be required to expose the set screw for adjustment. The cap-washer-spring mechanism may be adjusted by substituting different caps, washers and/or springs. Caps and washers with more pronounced ridges, or stiffer springs, would increase the overload threshold, while relatively shallow ridges, or softer springs, would decrease the overload threshold. Ring 4200 may be replaced with different rings having stiffer or more compliant beams or differently-shaped heads. The depth, diameter, and shape of any of the dimples disclosed herein may be modified to adjust the system response to overload, although such adjustment may involve exchanging component parts of the prosthetic connector.

Easily-adjusted embodiments may be adjusted by the patient using simple tools, such as a hex key or screwdriver. These embodiments may be desirable when frequent adjustments are anticipated. Embodiments that require partial disassembly or component substitution for adjustment may be adjusted by a prosthetist rather than by the patient. These embodiments may be desirable when frequent adjustment is not anticipated or to prevent unauthorized adjustment.

Prosthetic connectors are contemplated which are capable of dispersing some of the energy from an overload condition. In one example, a prosthetic connector may include one or more frangible elements which break upon overload. The frangible elements may experience a predetermined amount of elongation or bending prior to fracture, thus absorbing a portion of the overload energy. For instance, an axial shear pin could substitute for the axial ball, spring, and cap in an alternate version of prosthetic connector 100. In another example, a bushing may incorporate a crumple zone to absorb overload energy. The crumple zone may, for instance, be located around a central aperture in the bushing so that a shaft extending through the aperture crushes a portion of the bushing as the shaft is forced sideways by an overload condition. In yet another example, a prosthetic connector may incorporate a fluid damper in order to absorb some of the overload energy.

The prosthetic connectors disclosed herein may also include an alert or alarm system to let the user know that an overload condition may be imminent. In one example, the dimples of a prosthetic coupler may be modified to include an intermediate edge, or ridge, over which the corresponding ball must ride before fully exiting the dimple. More specifically, the ball would ride over the ridge and generate an alert or alarm at a load which is less than the overload. This design may produce an audible and/or tactile warning click as the ball rides over the ridge. In this design, the increment between the alarm load and the overload is built into the dimple so that adjustments to the overload setting would also adjust the alarm load. Thus, the alarm load may be considered to be pre-set in relation to the magnitude of the overload. In another example, a prosthetic connector may include one or more sensors proximate the ball and integrated with an alarm component. The sensor may react to slight movement of the ball out of the corresponding dimple, sending a signal to the alarm component, which may produce a sound or vibration to warn the user. Such a design may incorporate software, gages, buzzers, bells, or other components. In yet another example, the sensor or gage may react to pressure between the ball and dimple rather than frank movement, in order to provide an earlier warning to the patient. Similar designs are contemplated for embodiments that use the ridged washer, ring with beam, or equivalents.

The prosthetic connectors disclosed herein may be used in other applications. For example, the prosthetic connectors may be used in conjunction with an osseointegrated percutaneous prosthetic mounting system or direct skeletal attachment system. The prosthetic connectors may also be used in conjunction with a socket type prosthetic system, in order to manage the forces transmitted to the stump soft tissue interface.

The present invention may be embodied in other specific forms without departing from its spirit or essential characteristics. It is appreciated that individual features or structures selected from different examples described above may be combined together to form a variety of additional examples. As such, the described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. An apparatus for coupling a prosthetic limb to a residual limb attachment system, comprising:
a prosthetic limb connector for firmly connecting the apparatus to the prosthetic limb; and
a residual limb connector for firmly connecting the apparatus to the residual limb attachment system;
wherein the apparatus comprises a physiological configuration, in which the prosthetic limb connector is axially aligned, rotationally aligned, and firmly fixed relative to the residual limb connector, and
an overload configuration, in which the prosthetic limb is out of alignment relative to the residual limb attachment system, wherein the apparatus transforms from the physiological configuration to the overload configuration in response to a predetermined applied load.

2. The apparatus of claim 1, wherein the apparatus is adjustable to respond to a selected predetermined applied load.

3. The apparatus of claim 1, wherein the apparatus automatically returns to the physiological configuration when the prosthetic limb connector is urged into alignment with the residual limb connector.

4. The apparatus of claim 1, comprising:
a bending clutch that responds to a predetermined applied bending load; and
a tension clutch that responds to a predetermined applied tension load;
wherein, in the physiological configuration, the bending clutch firmly fixes the prosthetic limb connector in axial alignment relative to the residual limb connector, and
the tension clutch firmly fixes the prosthetic limb connector in axial displacement relative to the residual limb connector; and
wherein, in the overload configuration, the bending clutch releases the prosthetic limb from axial alignment relative to the residual limb attachment system, and
the tension clutch releases the prosthetic limb to move axially away from the residual limb attachment system.

5. The apparatus of claim 1, comprising:
a bending clutch that responds to a predetermined applied bending load; and
a torsion clutch that responds to a predetermined applied torsion load;
wherein, in the physiological configuration, the bending clutch firmly fixes the prosthetic limb connector in axial alignment relative to the residual limb connector, and
the torsion clutch firmly fixes the prosthetic limb connector in rotational alignment relative to the residual limb connector; and
wherein, in the overload configuration, the bending clutch releases the prosthetic limb from axial alignment relative to the residual limb attachment system, and
the torsion clutch releases the prosthetic limb from rotational alignment relative to the residual limb attachment system.

6. The apparatus of claim 1, comprising:
a torsion clutch that responds to a predetermined applied torsion load; and
a tension clutch that responds to a predetermined applied tensile load;
wherein, in the physiological configuration, the torsion clutch firmly fixes the prosthetic limb connector in rotational alignment relative to the residual limb connector, and
the tension clutch firmly fixes the prosthetic limb connector in axial displacement relative to the residual limb connector; and
wherein, in the overload configuration, the torsion clutch releases the prosthetic limb from rotational alignment relative to the residual limb attachment system, and
the tension clutch releases the prosthetic limb to move axially away from the residual limb attachment system.

7. The apparatus of claim 4, wherein each clutch independently releases the prosthetic limb in response to the corresponding predetermined applied load.

8. The apparatus of claim 4, wherein both clutches release the prosthetic limb in response to one of the predetermined applied loads.

9. The apparatus of claim 4, wherein the bending clutch comprises:
a spring plunger mounted to a support structure, wherein the support structure is coupled to a selected one of the prosthetic limb connector and the residual limb connector; and
a sphere with a dimple, wherein the sphere is coupled to a remaining one of the prosthetic limb connector and the residual limb connector;
wherein the spring plunger presses against the dimple in the physiological configuration;
wherein the spring plunger rests outside the dimple against the sphere in the overload configuration.

10. The apparatus of claim 4, wherein the bending clutch comprises:
a cantilever beam plunger mounted to a support structure, wherein the support structure is coupled to a selected one of the prosthetic limb connector and the residual limb connector; and
a sphere with a dimple, wherein the sphere is coupled to a remaining one of the prosthetic limb connector and the residual limb connector;
wherein the cantilever beam plunger presses against the dimple in the physiological configuration;
wherein the cantilever beam plunger rests outside the dimple against the sphere in the overload configuration.

11. The apparatus of claim 5, wherein the torsion clutch comprises:
a spring plunger mounted to a support structure, wherein the support structure is coupled to a selected one of the prosthetic limb connector and the residual limb connector; and
a socket with a dimple, wherein the socket is coupled to a remaining one of the prosthetic limb connector and the residual limb connector;
wherein the spring plunger presses against the dimple in the physiological configuration;
wherein the spring plunger rests outside the dimple against the socket in the overload configuration.

12. The apparatus of claim 5, wherein the torsion clutch comprises:
a spring plunger mounted to a support structure, wherein the support structure is coupled to a selected one of the prosthetic limb connector and the residual limb connector; and
a sphere with a dimple, wherein the sphere is coupled to a remaining one of the prosthetic limb connector and the residual limb connector;
wherein the spring plunger presses against the dimple in the physiological configuration;
wherein the spring plunger rests outside the dimple against the sphere in the overload configuration.

13. The apparatus of claim 5, wherein the torsion clutch comprises:
a spring-loaded washer mounted to a support structure, wherein a first side of the washer comprises alternating radial ridges and grooves, wherein the support structure is coupled to a selected one of the prosthetic limb connector and the residual limb connector; and
a cap, wherein a first side of the cap comprises alternating radial ridges and grooves, wherein the cap is coupled to a remaining one of the prosthetic limb connector and the residual limb connector;
wherein the ridges and grooves of the washer interdigitate with the ridges and grooves of the cap in the physiological configuration;
wherein the ridges of the washer are outside the ridges of the cap in the overload configuration.

14. The apparatus of claim 4, wherein the tension clutch comprises a high friction interface between a selected one of the prosthetic limb connector and the residual limb connector, and a corresponding limb.

15. The apparatus of claim 4, wherein the tension clutch comprises a snap fit between a selected one of the prosthetic limb connector and the residual limb connector, and a corresponding limb.

16. The apparatus of claim 5, wherein each clutch independently releases the prosthetic limb in response to the corresponding predetermined applied load.

17. The apparatus of claim 5, wherein both clutches release the prosthetic limb in response to one of the predetermined applied loads.

18. The apparatus of claim 5, wherein the bending clutch comprises:
a spring plunger mounted to a support structure, wherein the support structure is coupled to a selected one of the prosthetic limb connector and the residual limb connector; and
a sphere with a dimple, wherein the sphere is coupled to a remaining one of the prosthetic limb connector and the residual limb connector;
wherein the spring plunger presses against the dimple in the physiological configuration;
wherein the spring plunger rests outside the dimple against the sphere in the overload configuration.

19. The apparatus of claim 5, wherein the bending clutch comprises:
a cantilever beam plunger mounted to a support structure, wherein the support structure is coupled to a selected one of the prosthetic limb connector and the residual limb connector; and
a sphere with a dimple, wherein the sphere is coupled to a remaining one of the prosthetic limb connector and the residual limb connector;
wherein the cantilever beam plunger presses against the dimple in the physiological configuration;
wherein the cantilever beam plunger rests outside the dimple against the sphere in the overload configuration.

20. The apparatus of claim 6, wherein each clutch independently releases the prosthetic limb in response to the corresponding predetermined applied load.

20. The apparatus of claim 6, wherein both clutches release the prosthetic limb in response to one of the predetermined applied loads.

22. The apparatus of claim 6, wherein the torsion clutch comprises:
a spring plunger mounted to a support structure, wherein the support structure is coupled to a selected one of the prosthetic limb connector and the residual limb connector; and
a socket with a dimple, wherein the socket is coupled to a remaining one of the prosthetic limb connector and the residual limb connector;
wherein the spring plunger presses against the dimple in the physiological configuration;
wherein the spring plunger rests outside the dimple against the socket in the overload configuration.

23. The apparatus of claim 6, wherein the torsion clutch comprises:
a spring plunger mounted to a support structure, wherein the support structure is coupled to a selected one of the prosthetic limb connector and the residual limb connector; and
a sphere with a dimple, wherein the sphere is coupled to a remaining one of the prosthetic limb connector and the residual limb connector;
wherein the spring plunger presses against the dimple in the physiological configuration;
wherein the spring plunger rests outside the dimple against the sphere in the overload configuration.

24. The apparatus of claim 6, wherein the torsion clutch comprises:
a spring-loaded washer mounted to a support structure, wherein a first side of the washer comprises alternating radial ridges and grooves, wherein the support structure is coupled to a selected one of the prosthetic limb connector and the residual limb connector; and
a cap, wherein a first side of the cap comprises alternating radial ridges and grooves, wherein the cap is coupled to a remaining one of the prosthetic limb connector and the residual limb connector;
wherein the ridges and grooves of the washer interdigitate with the ridges and grooves of the cap in the physiological configuration;
wherein the ridges of the washer are outside the ridges of the cap in the overload configuration.

25. The apparatus of claim 6, wherein the tension clutch comprises a high friction interface between a selected one of the prosthetic limb connector and the residual limb connector, and a corresponding limb.

26. The apparatus of claim 6, wherein the tension clutch comprises a snap fit between a selected one of the prosthetic limb connector and the residual limb connector, and a corresponding limb.
